(19) United States
(12) Patent Application Publication
(10) Pub. No.: US 2011/0028727 A1
(43) Pub. Date: Feb. 3, 2011

(54) PROCESS FOR THE SELECTIVE PREPARATION OF 3-METHYLPYRIDINE (3-PICOLINE) FROM ACROLEIN AND ONE OR MORE AMMONIUM SALTS DISSOLVED IN WATER

(76) Inventors: Wolfgang WENGER, Visp (CH); Andreas HEYL, Termen (CH); Lothar OTT, Visp (CH); Herbert VOGEL, Nauheim (DE); Paul HANSELMANN, Brig-Glis (CH); Gökhan ARAS, Babenhausen (DE)

Correspondence Address: HOFFMANN & BARON, LLP, 6900 JERICHO TURNPIKE, SYOSSET, NY, 11791, US

(21) Appl. No.: 12/844,267

(22) Filed: Jul. 27, 2010

Related U.S. Application Data
(60) Provisional application No. 61/229,585, filed on Jul. 29, 2009.

(30) Foreign Application Priority Data
Jul. 29, 2009 (EP) 09009823.7

(51) Int. Cl. C07D 213/12

(52) U.S. Cl. 546/251

(57) ABSTRACT

The present invention relates to a process for the selective preparation of 3-methylpyridine, characterized in that acrolein and one or more ammonium salt(s) dissolved in water are reacted continuously under high pressures and at temperatures of 200-400° C.

### CROSS-REFERENCE TO RELATED APPLICATIONS AND CLAIM OF PRIORITY

This application claims the benefit of priority from European Patent Application No. 09009823.7 filed Jul. 29, 2009, and U.S. Provisional Patent Application No. 61/229,585 filed Jul. 29, 2009, the disclosures of which are incorporated herein by reference.

### BACKGROUND OF THE INVENTION

The present invention relates to a process for the selective and continuous preparation of 3-methylpyridine from acrolein.

A substantial proportion of 3-methylpyridine is used as a starting material for the preparation of insecticides, e.g. chlorpyrifos, or drugs. It can also be used in the production of food supplements, such as nicotinic acid or nicotinamide, and herbicides, such as fluazifopbutyl [Shi2005].

According to the prior art, acrolein can be prepared, inter alia, from the dehydration of glycerol, which occurs as a “waste product” in relatively large amounts in biodiesel production, in near-critical and supercritical water with addition of sulphuric acid [Wat2007] or salts [Ott2006]. The direct addition of ammonia water or of a substance delivering ammonia under the reaction conditions to this starting mixture does not lead to the desired yields of 3-methylpyridine. The acrolein obtained is therefore subsequently converted into 3-methylpyridine in a further step.

It is known that 3-methylpyridine forms in the reaction of acrolein with ammonia in the gas phase in the presence of catalysts. Catalysts used are chiefly substances based on oxides and silicates of aluminium. Aluminium silicates which contain fluorosilicic acid or fluoroboric acid and have been pretreated by heating to 450° C. (DT-A 1 917 037) or zeolite molecular sieves having contents of lanthanum (DT-A 2 023 158) are used. The low space-time yields of these processes are disadvantageous.

In addition to 3-methylpyridine, a considerable amount of pyridine forms with the use of catalysts which consist of compounds pretreated at temperatures of 550 to 1200° C. with oxygen and comprising the elements Al, F and O and at least one element of the second, third and fourth group of the Periodic Table of the Elements (DE-A 2 151 417) or comprising at least two elements of the second, fourth, fifth and sixth group of the Periodic Table of the Elements (DE-A 2 224 160) or at least one element of the second group of the Periodic Table of the Elements (DE-A 2 239 801).

3-Methylpyridine is also obtained in low yields in the preparation of acrolein, propionaldehyde and ammonia with catalysts consisting of aluminium oxide, silicon oxide and optionally additions of oxides of further elements (French Patent 1 273 826). By the use of colloidal aluminium silicates, the yield of 3-methylpyridine can be increased to about 60% (DE-A 2 703 070).

Crystalline zeolites, having a silicon to aluminium ratio of at least 12 and a constraint index of 1 to 12, e.g. ZSM5, are used in U.S. Pat. No. 4,220,783 for the reaction of C2-C4 aldehydes, C3-C5 ketones or mixtures of said aldehydes and/or ketones with ammonia in the presence of methanol or water. A short life of the catalyst and low yields of pyridine and 3-picoline are disadvantages of this process. By the use of synthetic, porous and crystalline materials, e.g. zeolite MCM-22 or MCM-49, as a catalyst, the yields of pyridine and 3-alkylypyridines can be increased (U.S. Pat. No. 5,395,940). Here, formaldehyde, C2-C4 aldehydes, C3-C5 ketones or mixtures of said aldehydes and/or ketones, and ammonia and hydrogen, are used as reactants.

DE 3 634 259 discloses that a mixture of acrolein and alkanals is reacted with ammonia in the presence of a zeolite of the pentasil type selectively to give 3-methyl-pyridine without relatively large amounts of pyridine inevitably occurring. A yield of 91% of 3-methylpyridine can be obtained over 6 hours in a tubular reactor by reacting acrolein and propionaldehyde with ammonia in a three-fold stoichiometric excess. The reaction temperature is 400° C. and regeneration of the catalyst is necessary. It is also known that acrolein can be reacted with ammonium salts in a batchwise procedure, in an acidic reaction medium, e.g. propionic acid, and at temperatures of 15-150° C. (British Patent 1 240 928). The yields of 3-methylpyridine are relatively low at about 33%.

The preparation of 3-methylpyridine from acrolein or a mixture of acrolein and formaldehyde or a mixture of acrolein, formaldehyde and acetaldehyde in the liquid, aqueous phase at temperatures of 180-280° C. in a closed vessel in the presence of ammonia and/or ammonium salts, such as, for example, diammonium hydrogen phosphate, is disclosed in EP 0 075 727 and in Grayson, J. I. and Dinkel, R. “An Improved Liquid-Phase Synthesis of Simple Alkylpyridines” Helvetica Chimica Acta, Vol. 67 (1984), 2100-2110. After the process, 3-methylpyridine is obtained in yields of less than 60% and the formation of pyridine is substantially suppressed. The long times of 20-90 minutes for the addition of the aldehyde to the reaction solution are disadvantageous.

### DESCRIPTION OF THE INVENTION

The technical problem to be solved consisted in reacting the produced acrolein in a second stage continuously in high yields without use of catalysts and with short residence times to give 3-methylpyridine. This problem is solved by the process according to the invention, which is characterized in that acrolein and one or more ammonium salt(s) dissolved in water are reacted continuously under high pressures and at temperatures of 200-400° C.

The process according to the invention preferably takes place in a pH range of 4-8, particularly preferably of 4-6.

It is particularly preferred according to the invention to carry out the reaction in an acidic reaction medium, with the result that the formation of metal hydroxides and/or polymerization reactions of the acrolein can be prevented.

Inorganic ammonium salts, in particular ammonium sulphate, ammonium acetate and ammonium dihydrogen phosphate, are particularly preferred.

Under the reaction conditions according to the invention, the ammonium salts give ammonia, which is reacted with acrolein with formation of a heterocycle. It was surprisingly found that almost exclusively 3-methylpyridine is formed, but not pyridine and/or further pyridine derivatives, which would have to be separated from the desired product by subsequent complicated work-up steps.

The process according to the invention achieves a maximum 3-picoline yield of 35-60%, based on the starting compounds used.

It may be preferable according to the invention to obtain the acrolein from glycerol by means of processes known in the prior art.

Acetaldehyde and formaldehyde are obtained as main by-products of the process according to the invention. This mixture can be used, for example, as a starting substance for recovering acrolein and/or for the preparation of 3-methylpyridine.

The process according to the invention can be carried out both directly with the acrolein-containing reaction mixture of the acrolein synthesis step and with acrolein purified beforehand.

Depending on the density of the medium, according to the invention residence times of preferably 5-400 s, particularly preferably 150-300 s are established.

According to the invention, the reactions preferably take place at not more than 400° C. and 40 MPa.

The process according to the invention can be carried out in standard high-pressure units. Here, a unit having a flow-tube reactor comprising Inconel625 and a reactor volume of 4-50 ml is preferred. These starting mixtures are transported via two preheated, separate trains at not more than 35 ml min⁻¹ into the reactor.

### BRIEF DESCRIPTION OF THE DRAWINGS

FIG. 1 is a graph which shows yields of 3-methylpyridine and acetaldehyde in the continuous reaction of 0.25% (g g⁻¹) of acrolein with 1.03% (g g⁻¹) of ammonium dihydrogen phosphate in near-critical water at 360° C. and 30 MPa and with different residence times.

FIG. 2 is a graph which depicts yields of 3-methylpyridine and acetaldehyde in the continuous reaction of 0.25% (g g⁻¹) of acrolein with 0.59% (g g⁻¹) of ammonium sulphate in near-critical water at 250° C. and 30 MPa and with different residence times; and

FIG. 3 is a graph which also shows yields of 3-methylpyridine in the continuous reaction of 0.25% (g g⁻¹) of acrolein with 0.59% (g g⁻¹) of ammonium sulphate in water at 250° C. and with different pressures and residence times.

### EXAMPLES

The invention is explained by the following, nonlimiting examples.

### Example 1

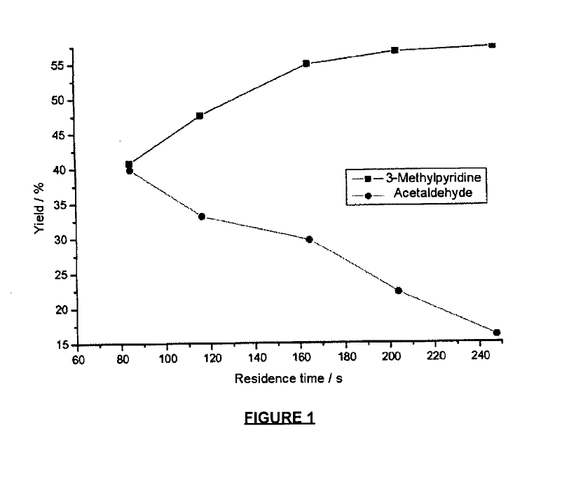

An aqueous solution comprising 0.75% (g g⁻¹) of acrolein and 3.07% (g g⁻¹) of ammonium dihydrogen phosphate, which corresponds to a molar ratio of acrolein to ammonium dihydrogen phosphate of 1:2, is reacted in a two-train high-pressure unit at 30 MPa. The liquid mixture is first heated to 170° C. in a preheating stage and then mixed with twice the amount of hot water, so that, at the reactor entrance of a tubular reactor comprising Inconel625 and having a volume of 49.5 ml, the reaction temperature is adjusted to 360° C. and near-critical water conditions prevail. The reaction solution is then cooled to room temperature in a heat exchanger and depressurized to atmospheric pressure. The liquid components are separated from the gaseous ones in a phase separator at 2° C. The liquid phase is collected and the fractions of the detectable components are determined by gas chromatography. For the quantitative determination of pyridine and its derivatives, the acidic sample solution is adjusted to a pH of 7-8 with ammonia water. 3,5-Dimethylpyridine, which was not detectable in preceding investigations, is used as an internal standard. For the determination of acetaldehyde and of acrolein, only 1-butanol is added to the sample as an internal standard. The yields of 3-methylpyridine and acetaldehyde determined under the conditions described are shown in FIG. 1. The maximum yield of 3-methylpyridine is 57% with a residence time of 248 s. Pyridine is obtained in insignificant amounts, with a yield of not more than 2%. Other pyridine derivatives are formed only in traces.

### Example 2

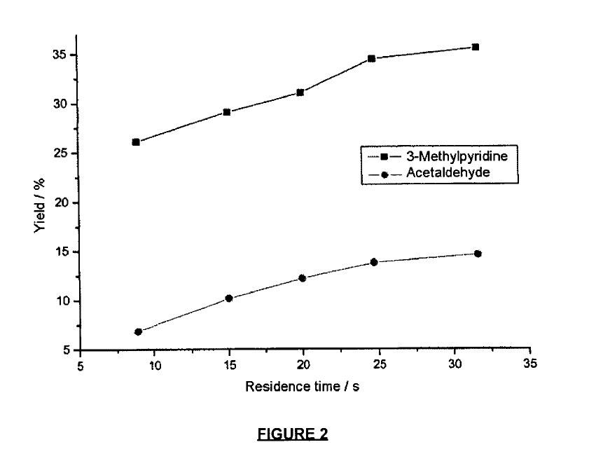

The reaction is effected with acrolein and ammonium sulphate in a molar ratio of 1:1. An aqueous solution of 0.75% (g g⁻¹) of acrolein is first heated to 50° C. in a preheating stage and then mixed with twice the amount of a preheated aqueous solution with 0.89% (g g⁻¹) of ammonium sulphate, so that a reaction temperature of 250° C. is established at the reactor entrance of a flow-tube reactor comprising Inconel625 and having a reactor volume of 4.4 ml. Depending on the volume flow rate and density of the reaction medium, residence times of 5-35 s are established. The results are shown in FIG. 2. The maximum yield of 3-methylpyridine is 36% with a residence time of 32 s. Pyridine is obtained with a yield of not more than 1%, other pyridine derivatives once again being formed only in traces.

### Example 3

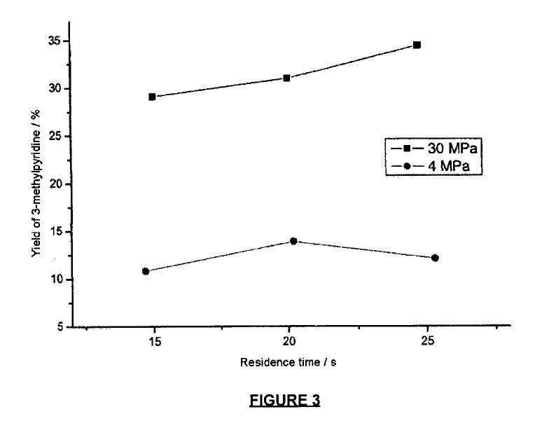

The reaction takes place according to Example 2. A pressure adjustment is carried out so that the operating pressure is slightly above the vapour pressure of the reaction solution. In FIG. 3, the yields of 3-methylpyridine at 4 MPa and 30 MPa are compared.

The maximum 3-methylpyridine yield of 14% at a low reaction pressure is substantially below the values obtained with the use of higher pressures.

### Example 4

An aqueous solution with 1.00% (g g⁻¹) of glycerol and 0.05% (g g⁻¹) of zinc sulphate is reacted according to Example 1 at 25 MPa. The liquid mixture is first heated to 230° C. in a preheating stage and then mixed with twice the amount of hot water, so that, at the reactor entrance, the reaction temperature of 360° C. is established and near-critical water conditions prevail. The residence time in the reactor is 140 s. All liquid components are detected by gas chromatography using N-methyl-2-pyrrolidone as an internal standard. At a 73% conversion of glycerol, acrolein and acetaldehyde are obtained with yields of 20% and 28%, respectively.

In a second reaction step, the reaction solution obtained from the first reaction step and having an acrolein concentration of 0.12% (g g⁻¹) is mixed with the equimolar amount of ammonia sulphate and reacted at 360° C. and 30 MPa and with a residence time of 160 s. Here, the starting material mixture is passed in undiluted form into the reactor. 3-Methylpyridine is obtained with a yield of 43%. The total yield of 3-methyl-pyridine, based on glycerol, is 8%.

### LITERATURE

[Ott2006] L. Ott, M. Bicker, H. Vogel: Catalytic dehydration of glycerol in sub- and supercritical water: a new chemical process for acrolein production, Green Chemistry, 2006, 8, 214-220.

[Shi2005] S. Shimizu, N. Watanabe, T. Kataoka, T. Shoji, N. Abe, S. Morishita, H. Ichimura: Pyridine and Pyridine Derivatives, Ullmann's Encyclopedia of Industrial Chemistry, 7th Edition, Wiley Interscience, Online Release, 2005.

[Wat2007] M. Watanabe, T. lida , Y. Aizawa, T. M. Aida, H. Inomata: Acrolein synthesis from glycerol in hot-compressed water, Bioresource Technology, 2007, 98, 1285-1290.

1. Process for the selective preparation of 3-methylpyridine, characterized in that acrolein and one or more ammonium salt(s) dissolved in water are reacted continuously under high pressures and at temperatures of 200-400° C. and the contact or residence time is 5-400 s, preferably 150-300 s.

2. Process according to claim 1, characterized in that ammonium sulphate, ammonium acetate and/or ammonium dihydrogen phosphate are used.

3. Process according to claim 1, characterized in that the acrolein and the ammonium salt are used in a molar ratio of 1:0.125 to 1:2.

4. Process according to claim 1, the pressure being adjusted so that the reaction mixture is present in liquid single-phase form.

5. Process according to claim 1, characterized in that the reaction is effected at pressures of 20-40 MPa.

6. Process according to claim 1, the pH of the aqueous solution being in a range of 4-8.

7. Process according to claim 1, the yield of 3-picoline being 35-60%, based on the starting compounds used.

8. Process according to claim 1, acetaldehyde and formaldehyde being obtained as by-products and optionally being recycled to the process.

9. Process according to claim 8, the yield of acetaldehyde being between 35 and 50%, based on the starting compounds used.

10. Process according to claim 1, the acrolein being obtained from glycerol.
